(19) United States
(12) Patent Application Publication
(10) Pub. No.: US 2016/0061807 A1
(43) Pub. Date: Mar. 3, 2016

(54) SPECTRAL SIGNATURE DRUG DETECTION

(71) Applicants: Lifeloc Technologies, Inc., Wheat Ridge, CO (US); United States Naval Research Laboratory, Washington, DC (US)

(72) Inventors: Gurumurthi Ravishankar, Englewood, CO (US); R. Andrew McGill, Lorton, VA (US); Jim R. Smith, Westminster, CO (US); Viet Nguyen, Gaithersburg, MD (US); Robert Furstenberg, Burke, VA (US); Brandon Wellborn, Arvada, CO (US)

(21) Appl. No.: 14/837,487

(22) Filed: Aug. 27, 2015

Related U.S. Application Data
(60) Provisional application No. 62/042,667, filed on Aug. 27, 2014.

(51) Int. Cl. G01N 33/487; G01N 21/3577

(52) U.S. Cl. CPC G01N 33/487; G01N 21/3577

(57) ABSTRACT

The technology disclosed herein may be used to detect drugs with potential for abuse within a human subject. This technology may be particularly useful to discriminate between drugs of abuse, corresponding psychoactive compounds, and corresponding metabolite byproducts, which are often closely related and possess similar chemical structures. The disclosed technology uses infrared light reflectance characteristics particular to one or more chemical compounds to be detected for identification of those compounds within the human subject.

### CROSS-REFERENCE TO RELATED APPLICATIONS

The present application claims benefit of priority to U.S. Provisional Patent Application No. 62/042,667, entitled “Drug Detection Using Infrared Light” and filed on Aug. 27, 2014, which is specifically incorporated by reference herein for all that it discloses or teaches.

### STATEMENT REGARDING FEDERALLY SPONSORED RESEARCH OR DEVELOPMENT

This invention was made with government support under NCRADA-NRL-13-534 awarded by The Naval Research Laboratory (NRL). The government has certain rights in the invention.

### BACKGROUND

Drug abuse is costly to society in terms of increased healthcare cost, lost productivity, loss of life, and property damage, for example. Rapid detection of drugs, both legal and illegal, with potential for abuse and/or their corresponding psychoactive compounds within the human body is useful to monitor, deter, and/or reduce drug abuse. A suite of relatively compact and portable devices (e.g., handheld devices) and bench-top devices for breath and blood specimens exists for alcohol detection, monitoring, and measurement within the human body. These devices have been deployed by law enforcement and in workplace environments for decades, with proven results. However, the development of similarly compact, portable, and reliable devices for detecting other drugs with potential for abuse has proven more elusive.

At least two challenges for detecting and quantifying various drugs within a human subject are: 1) the relatively low concentration levels present in the human subject after ingestion, inhalation, injection, or other form of entry of the drug into the human subject; and 2) the relative difficulty in discriminating between psychoactive (or parent) drug compounds, which cause impairment of normal activities (e.g., driving) and byproducts or metabolites produced within the body, which may or may not be psychoactive. Liquid or gas chromatography may be capable of meeting these challenges, but is generally limited to use in laboratory environments by trained scientists and is time-consuming and/or costly.

Marijuana, for example, is in the midst of a shift from illegal to legal status across the United States and elsewhere around the world. With marijuana's changing legal status brings increased availability and potentially increased risk of abuse, with potentially broad adverse societal impacts. Delta-9-tetrahydrocannabinol (THC) is the primary psychoactive compound responsible for marijuana intoxication, however, the Delta-9-THC level present in a human subject after ingestion of cannabis smoke or cannabis-infused edibles is quite low, often ranging from several nanograms per mL, in the case of blood or saliva, to several picograms per square inch for exhaled breath condensate. Furthermore, the human subject rapidly metabolizes the Delta-9-THC to 11-Hydroxy-Delta-9-THC and 11-nor-9-Carboxy-Delta-9-THC, which possess very similar chemical structures as the Delta-9-THC, but have reduced or non-existent psychoactive effects on the human subject by comparison to Delta-9-THC. There is a need for new methods and devices to detect drugs with the potential for abuse which address some or all of the aforementioned challenges.

### SUMMARY

Implementations described and claimed herein address the foregoing problems by providing an infrared drug detector comprising: a bodily fluid collector directed at a discrete location on a substrate and configured to deposit a bodily fluid specimen on the substrate; an infrared source directed at the discrete location on the substrate and configured to emit a source beam at the bodily fluid specimen; and an infrared detector configured to receive a spectral signature of the bodily fluid specimen following interaction of the bodily fluid specimen with the infrared source beam to detect the presence of an analyte within the bodily fluid specimen.

Implementations described and claimed herein address the foregoing problems by further providing a method comprising: depositing a bodily fluid specimen at a discrete location on a substrate; directing an infrared source beam at the discrete location on the substrate; detecting a spectral signature of the bodily fluid specimen on the substrate following interaction of the bodily fluid specimen with the infrared source beam; and identifying one or more analytes within the bodily fluid specimen using the detected spectral signature.

Implementations described and claimed herein address the foregoing problems by still further providing a method of infrared drug detection comprising: directing an infrared source beam at a discrete location on a substrate; detecting a control spectral signature of the substrate following interaction of the substrate with the infrared source beam; depositing a bodily fluid specimen at the discrete location on the substrate following detection of the control spectral signature of the substrate; directing the infrared source beam at the discrete location on the substrate following deposition of the bodily fluid specimen; detecting a condensate spectral signature of the bodily fluid specimen on the substrate following interaction of the bodily fluid specimen with the source beam; and identifying one or more analytes within the bodily fluid specimen using the detected spectral signature.

Other implementations are also described and recited herein.

### BRIEF DESCRIPTIONS OF THE DRAWINGS

FIG. 1A illustrates a control detection process using an example infrared (IR) drug detection device.

FIG. 1B illustrates a drug detection process using the example IR drug detection device of FIG. 1A.

FIG. 2 is a block diagram of an example IR drug detection device.

FIG. 3 illustrates the chemical structures for tetrahydrocannabinolic acid (THCA) and three of its analytes that typically occur when the THCA is used as a drug.

FIG. 4 illustrates an example breath condensate collection device.

FIG. 5 illustrates a schematic of an example IR drug detection device.

FIG. 6A is an example graph of IR absorbance as a function of wavelength for a drug analyte.

FIG. 6B is an example graph of IR reflectance as a function of wavelength for a drug analyte.

FIG. 7 illustrates example operations for using an IR drug detection device to detect the presence of one or more analyte(s) in a test specimen.

### DETAILED DESCRIPTIONS

The presently disclosed technology provides devices and methods for detection, discrimination, and quantification of one or more analytes (e.g., a drug or psychoactive compound) in a test specimen. The test specimen could include one or more of blood (or blood components), saliva, perspiration, lacrimation, urine, and breath aerosol or condensate, for example. The disclosed technology is not limited to detection of a specific class or type of drug. For example, the disclosed technology can be used to detect analytes from multiple types or classes of drugs (e.g., the Substance Abuse and Mental Health Services Administration (SAMHSA) 5, which includes opiates, amphetamines, cocaine, cannabinoids, and phencyclidine). At least the following drugs of abuse may be identified in breath condensate specimens: alcohol, methadone, amphetamine, methamphetamine, 6-acetylmorphine, morphine, benzoylecgonine, cocaine, diazepam, oxazepam, alprazolam, buprenorphine, and Delta-9-THC using the presently disclosed technology. In an example implementation, the disclosed technology may be used to detect one or more analytes among these chemical compounds from a subject's breath specimen.

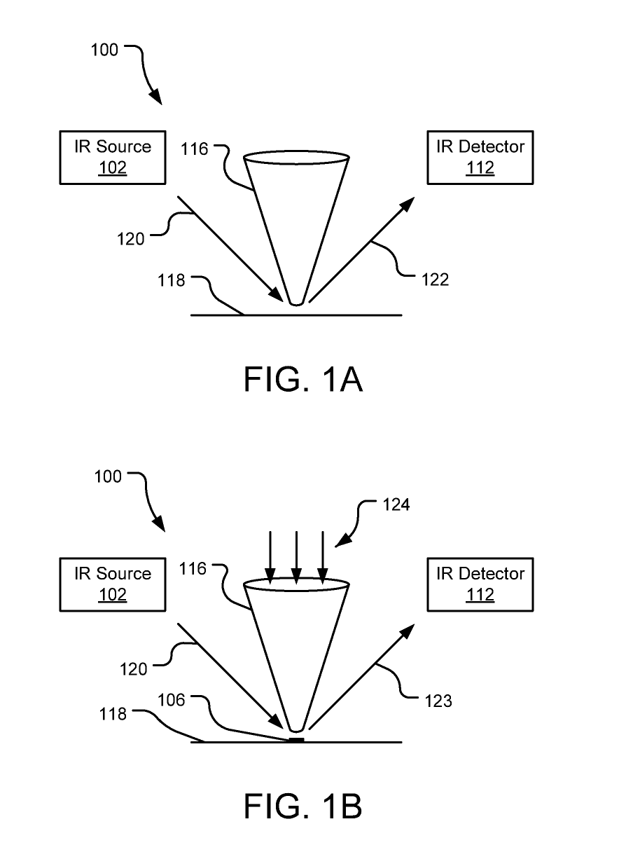

FIG. 1A illustrates a control detection process using an example IR drug detection device 100. The device 100 may be packaged as a portable device for use by law enforcement or other personnel to quickly and easily collect and analyze a test specimen (not shown, see test specimen 106 of FIG. 1B) for the presence of one or more drug or other chemical analytes. The device 100 includes a collection component 116 (e.g., a bodily fluid, saliva or breath condensate collector), which collects the test specimen 106 and directs it to a specific discrete location on a substrate 118.

The device 100 further includes an IR source 102, which may utilize any available IR generating technology (e.g., broadband, laser, tunable, non-tunable, pulsed, continuous wave, etc.). Further, the IR source 102 may include multiple individual IR sources (e.g., operating in a multi-spectral mode) or a single tunable IR source (e.g., operating in a hyper-spectral mode). Such IR sources may impart greater selectivity and analyte discriminating ability to the device 100. Still further, the IR source 102 may be eye-safe to protect humans in close physical proximity to the device 100. In an example implementation, the IR source 102 includes a set of fixed-wavelength quantum cascade lasers (QCLs), with each wavelength in the set selected to exploit differences in IR spectral features amongst various compounds present in the test specimen. In another example implementation, a tunable wavelength QCL may be used in a similar fashion for the IR source 102.

In various implementations, the IR source 102 operates in the near-IR (i.e., approximately 14000 cm⁻¹-4000 cm⁻¹), mid-IR (i.e., approximately 4000 cm⁻¹-400 cm⁻¹), or far-IR (i.e., approximately 400 cm⁻¹-10 cm⁻¹) range. In other implementations, the IR source 102 is replaced with a radiant source operating in a non-IR spectrum (e.g., the visible or ultra-violet spectrums). As a result, the remaining components of the device 100 are adapted to work with the radiant spectrum emitted by the radiant source.

A source beam 120 is directed at the substrate 118. In various implementations, portions of the source beam 120 are reflected from the substrate 118, absorbed by the substrate 118, and/or transmitted through the substrate 118. In the implementation of FIG. 1A, a portion of the source beam 120 is reflected from the substrate 118 to generate reflected beam 122, which has a wavelength-intensity pattern (or spectral signature) commensurate with the substrate 118 and its interaction with the source beam 120. An IR detector 112 receives the reflected beam 122. This is referred to herein as reflectance IR drug detection. In other implementations, the IR detector 112 is oriented to detect a portion of the source beam 120 that is transmitted through the substrate 118, which has a wavelength-intensity pattern (or spectral signature) commensurate with the substrate 118 and its interaction with the source beam 120. This is referred to herein as transmittance IR drug detection.

In still other implementations, a portion of the source beam 120 is absorbed by the substrate 118 to generate a thermal signature, which has an intensity pattern commensurate with the substrate 118, and its interaction with the source beam 120. The thermal signature is detected by a resonant photo-thermal detector (not shown), for example. This is referred to herein as absorbance IR drug detection. In an example absorbance IR drug detection implementation, the source beam 120 wavelength is tuned across IR absorption feature(s) of target analyte(s). Broadband IR emission, which corresponds to heat due to IR absorption by the analyte on the substrate 118, is detected and related to the identify and quantity of the analyte(s) on the substrate 118. Further, microscope objective optics (not shown) may be used in conjunction with the IR detector 112 to detect very low levels of Delta-9-THC (e.g., less than 50 nanograms), for example. Still further, photo-thermal detection can provide a specific analyte location within the specific discrete location on the substrate 118.

The IR detector 112 is one or more of an array of available IR detectors, including, but not limited to, a point detector, a linear detector, and a 2D-array detector, each of which may be temperature controlled in some implementations. The IR detector 112 detects and outputs a spectral signature of the substrate 118 (e.g., a mapping of the intensity of the reflected beam 122 as a function of wavelength). This mapping is used as a control pattern indicative of the substrate 118 without a test specimen thereon.

FIG. 1B illustrates a drug detection process using the example IR drug detection device 100 of FIG. 1A. A user of the device 100 may direct the test specimen 106 to the substrate 118 via the collection component 116 as illustrated by arrows 124. In one example implementation, the user places his/her mouth over the collection component 116 and blows a breath specimen through the collection component 116, where the test specimen 106 (e.g., an array of saliva droplets) is collected and retained on the substrate 118 in the specific discrete location where the collection component 116 directs the test specimen 106. In other implementations, the collection component 116 may otherwise collect saliva, or alternatively other bodily fluid as the test specimen 106.

The IR source 102 generates the source beam 120 that is directed at the substrate 118. In various implementations, the source beam 120 is reflected from the substrate 118, absorbed by the substrate 118, and/or transmitted through the substrate 118. In the reflectance implementation of FIG. 1B, a portion of the source beam 120 is reflected from the substrate 118 to generate reflected beam 123, which has a wavelength-intensity pattern (or spectral signature) commensurate with the test specimen 106 and the substrate 118 and their interaction with the source beam 120. The IR detector 112 receives the reflected beam 123. In a transmittance implementation, the IR detector 112 is oriented to detect a portion of the source beam 120 that is transmitted through the substrate 118, which has a wavelength-intensity pattern (or spectral signature) commensurate with the test specimen 106 and the substrate 118 and their interaction with the source beam 120. In an absorbance implementation, a portion of the source beam 120 is absorbed by the substrate 118 to generate a thermal signature, which has an intensity pattern commensurate with the test specimen 106 and the substrate 118 and their interaction with the source beam 120. The thermal signature is detected by a photo-thermal detector (not shown).

The IR detector 112 detects and outputs a spectral signature of the substrate 118 and the test specimen 106 (e.g., a mapping of the intensity of the reflected beam 123 as a function of wavelength). This mapping is compared with the mapping of the intensity of the reflected beam 122 of FIG. 1A to identify any features that are solely attributable to the test specimen 106 (i.e., screening out features attributable to the substrate 118). The features that are attributable to the test specimen 106 are then compared to known IR response characteristics of one of more analytes in order to detect possible presence of the analytes within the test specimen 106.

In an example implementation, the IR detector 112 relies on two distinct regions within the mid-IR range: 1) the ‘fingerprint region’ (wavelength ranging from 500 cm⁻¹-1500 cm⁻¹), where complex and closely spaced spectral features are found that are characteristic of the bending vibrational modes of the analyte molecules; and 2) the ‘functional group region’ (wavelength ranging from 1500 cm⁻¹-4000 cm⁻¹, which typically contains broader spectral features that are readily assigned to specific functional groups within the analyte molecule(s). In various implementations, the presently disclosed technology may utilize spectral features in one or both of the aforementioned mid-IR regions to detect and measure the presence of one or more analytes.

In some implementations, the device 100 may analyze the test specimen 106 without any physical contact with the test specimen 106, which could consume or otherwise significantly alter the test specimen 106. As a result, the test specimen 106 may be saved for future testing or evidentiary purposes and does not need particular preparation work done to it prior to performing drug detection operations (i.e., the drug detection operations are performed non-destructively on the test specimen 106). In other implementations, the device 100 consumes or alters a part of or the entire test specimen 106 as a consequence of the drug detection operations.

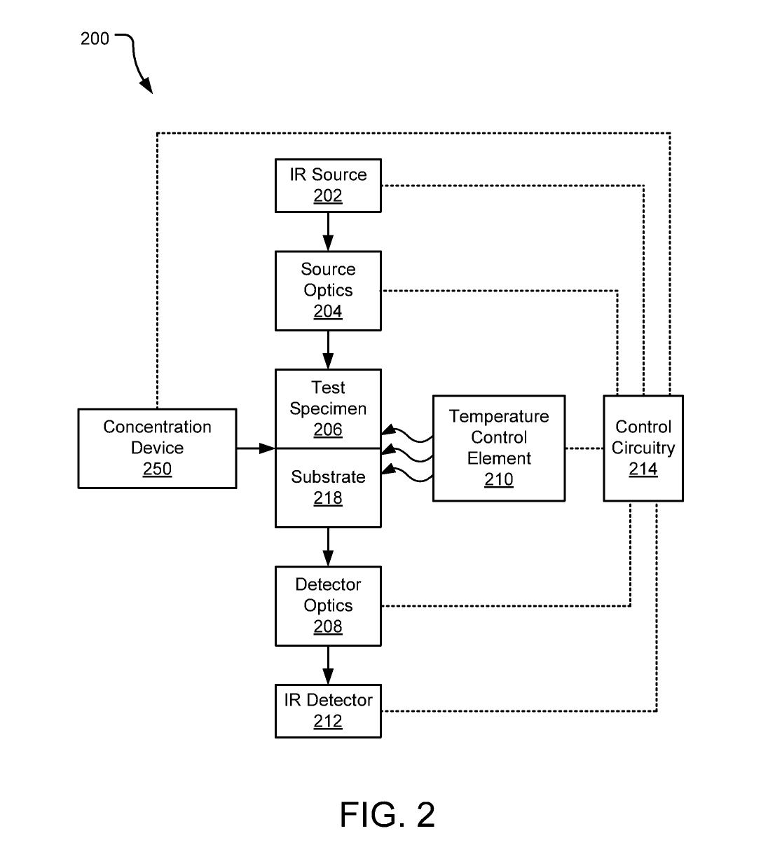

FIG. 2 is a block diagram of an example IR drug detection device 200. The device 200 may be packaged as a portable device for use by law enforcement or other personnel to quickly and easily analyze a test specimen 206 for the presence of one or more drug or other chemical analytes. The device 200 includes an IR source 202, which may utilize any available IR-generating technology and may include an array of multiple individual IR sources or a single IR source. In various implementations, the IR source 202 operates in the near-IR, mid-IR, or far-IR range. In other implementations, the IR source 202 is replaced with a radiant source operating in a non-IR spectrum. The remaining components on the device 200 are adapted to work with the radiant spectrum emitted by the radiant source.

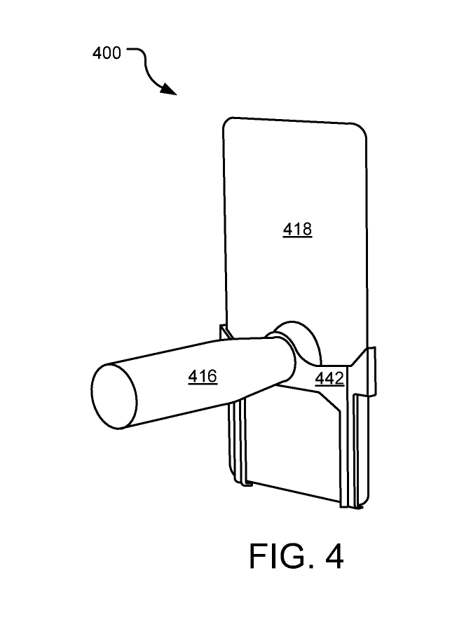

The device 200 further includes source optics 204, which may steer, shape, filter, and/or disperse the light emitted from the IR source 202. The source optics 204 may include, lenses, microscope objectives, mirrors, filters, diffraction gratings, prisms, choppers, and/or polarizers, for example. The source optics 204 direct a beam of the light emitted from the IR source 202 to the test specimen 206 deposited on a test substrate 218. In various implementations, a substrate holder (not shown, see e.g., substrate holder 442 of FIG. 4) may retain the substrate 218 and the test specimen 206 at a desired location on or within the device 200.

In some implementations, the substrate 218 and the test specimen 206 may be conductively connected to a temperature control element 210. The temperature control element 210 may heat and/or cool the test specimen 206 to reach or maintain a desired detection temperature at which the accuracy of the device 200 is best, or at least acceptable (e.g., 50° C.-100° C.). In an example implementation, the temperature control element 210 is a resistive heating element.

Further, a concentration device 250 may concentrate the test specimen 206 at a discrete location on the test substrate 218 prior to detecting the presence of one or more drug or other chemical analytes within the test specimen 206. In an example implementation, the concentration device 250 dissolves the test specimen 206 in an alcohol (e.g., methanol) and the alcohol entrained with the test specimen 206 is deposited at the discrete location on the test substrate 218. In some implementations, the alcohol quickly dissipates into the atmosphere leaving only the test specimen 206 remaining at the discrete location on the test substrate 218 for detecting the presence of one or more drug or other chemical analytes within the test specimen 206. In other implementations, the alcohol has a distinct spectral signature that can be distinguished from the spectral signature of the alcohol when the IR drug detection device 200 is used for detecting the presence of one or more drug or other chemical analytes within the test specimen 206.

In various implementations, portions of the source beam are reflected from the substrate 218, absorbed by the substrate 218, and/or transmitted through the substrate 218. In a reflectance implementation, a portion of the source beam is reflected from the substrate 218 to generate a reflected beam, which has a wavelength-intensity pattern (or spectral signature) commensurate with the test specimen 206 and the substrate 218 and their interaction with the source beam. The reflected beam is directed to detector optics 208. In a transmittance implementation, a portion of the source beam is transmitted through the substrate 218 to generate a transmitted beam, which has a wavelength-intensity pattern (or spectral signature) commensurate with the test specimen 206 and the substrate 218 and their interaction with the source beam. The transmitted beam is directed to the detector optics 208.

In an absorbance implementation, a portion of the source beam is absorbed by the substrate 218 to generate a thermal emission signature, which has an intensity pattern (or spectral signature) commensurate with the test specimen 206 and the substrate 218 and their interaction with the source beam. The thermal signature is detected by a photo-thermal detector (not shown). Detection of portions of the source beam reflected from the test specimen 206 and the substrate 218, absorbed by the test specimen 206 and the substrate 218, and/or transmitted through the test specimen 206 and the substrate 218 is referred to herein as detecting a spectral signature of the test specimen 206 and the substrate 218.

The detector optics 208 may steer, shape, filter, and/or collect the reflected or transmitted beam to an IR detector 212. The IR detector 212 may utilize any available IR detecting technology and may include an array of multiple individual IR detectors or a single IR detector. The IR detector 212 outputs a mapping of the intensity of the reflected or transmitted beam as a function of wavelength.

Control circuitry 214 electronically interconnects components of the device 100 (e.g., the IR source 202, the source optics 204, the detector optics 208, the IR detector 212, the temperature control element 210, and/or the concentration device 250) and provides input/output interface(s) for a user of the device 200. More specifically, the control circuitry 214 may provide control functionality, specimen testing automation, signal manipulation and processing, data acquisition, and result display functionality to the device 200. The control circuitry 214 may also control the temperature, humidity, and/or pressure within the device 200, depending upon the requirements of a particular implementation. The control circuitry 214 may include one or more processors, memory devices, modulating circuits, pre-amplifiers, amplifiers, input keys or touchscreens, and output displays.

The control circuitry 214 compares the mapping of the intensity of the reflected or transmitted beam as a function of wavelength with a similar mapping of the intensity of a control reflected or transmitted beam (i.e., a beam that interacted with the substrate 218 without the test specimen 206 thereon) to identify any features that are solely attributable to the test specimen 206 (i.e., screening out features attributable to the substrate 218). The control circuitry 214 then compares features that are attributable to the test specimen 206 to known IR response characteristics of one or more analytes in order to detect possible presence of the analytes within the test specimen 206.

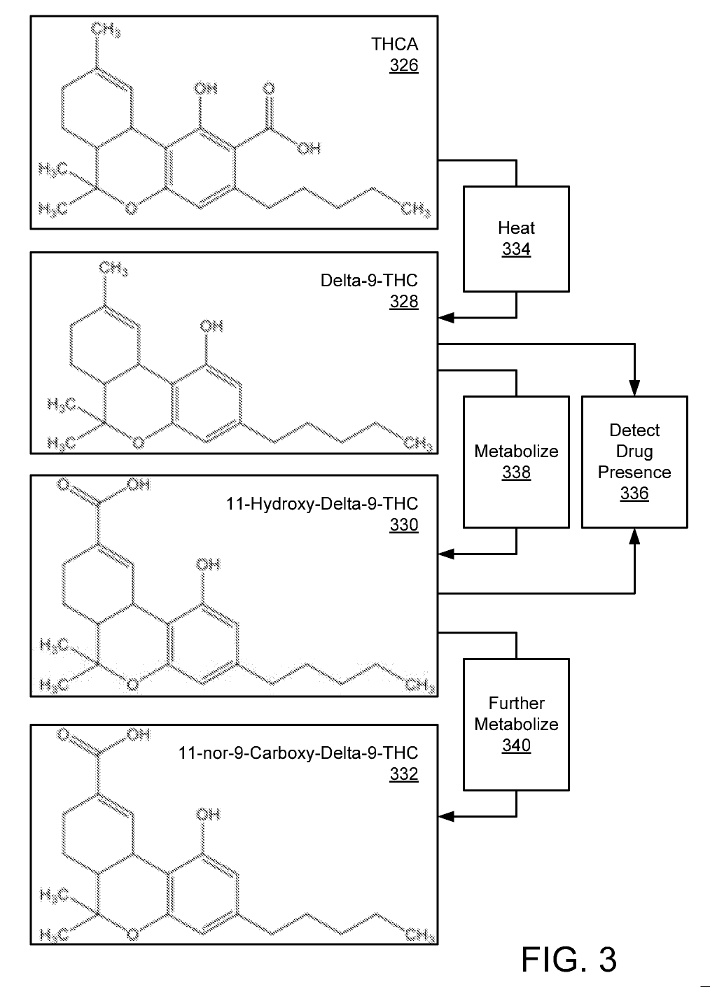

FIG. 3 illustrates THCA chemical structure 326 and three following chemical structures 328, 330, 332 that typically occur when the THCA 326 is used as a drug. THCA (alternatively, THC-A, tetrahydrocannabinolic acid, 2-COOH-THC, or other variants thereof) is a naturally-occurring chemical compound found in cannabis with a chemical structure as shown in FIG. 3. THCA is generally considered not psychoactive when consumed by a user. While the drug detection processes and devices disclosed herein are capable of detecting the presence of THCA in a human subject, its presence is generally ignored since THCA is not psychoactive. More specifically, the presence of THCA within the human subject is ignored because it is not a detriment to cognitive function of the human subject. In some implementations, the drug detection processes and devices disclosed herein are specifically set up such that THCA is not even detected if present within the human subject.

A heating operation 334 heats the THCA to a temperature exceeding 105 degrees Celsius, which causes the THCA to chemically change to a Δ9-THC (alternatively, delta-9-THC or variants thereof) structure 328. The Δ9-THC structure 328 is very similar to the THCA structure 326, however, Δ9-THC is psychoactive while the THCA is not psychoactive. The heating of the THCA is often accomplished by burning cannabis prior to ingestion by a user (e.g., inhaling, drinking, and/or eating the Δ9-THC). Detect drug presence operation 336 detects the presence of Δ9-THC in the human subject and distinguishes it from the THCA and other similar non-psychoactive THC compounds. Further, other drug detection processes and devices disclosed herein are capable of detecting the presence of Δ9-THC in the human subject and distinguishing it from THCA and other similar THC compounds.

After ingestion, metabolizing operation 338 metabolizes the Δ9-THC over time and yields the hydroxyl-Δ9-THC (alternatively, 11-hydroxy-delta-9-THC, 11-OH-THC, or other variants thereof) structure 330, which is similar to the Δ9-THC structure 328. While hydroxyl-Δ9-THC is also psychoactive, it may yield different psychoactive effects than the Δ9-THC on the human subject. The detect drug presence operation 336 also detects the presence of hydroxyl-Δ9-THC in the human subject and distinguishes it from THCA and other similar non-psychoactive THC compounds. In some implementations, the detect drug presence operation 336 may also distinguish between detected psychoactive THC compounds (e.g., Δ9-THC and hydroxyl-Δ9-THC). Further, other drug detection processes and devices disclosed herein are capable of detecting the presence of hydroxyl-Δ9-THC in the human subject and distinguishing it from THCA and other similar THC compounds.

Further metabolizing operation 340 further metabolizes the hydroxyl-Δ9-THC within the human subject and yields carboxy-Δ9-THC (alternatively, 11-nor-9-carboxy-delta-9-THC, THC-COOH, or other variants thereof) structure 332, which is similar to the hydroxyl-Δ9-THC structure 330. Liver cytochrome P450 enzymes CYP2C9, CYP2C19, and CYP3A4 primarily perform the metabolizing operations 338, 340. Carboxy-Δ9-THC is generally considered not psychoactive. While the drug detection processes and devices disclosed herein are capable of detecting the presence of carboxy-Δ9-THC in the human subject, its presence is generally ignored since carboxy-Δ9-THC is not psychoactive. More specifically, the presence of carboxy-Δ9-THC within the human subject is ignored because it does not impair cognitive function. In some implementations, the drug detection processes and devices disclosed herein are specifically set up such that carboxy-Δ9-THC is not even detected if present within the human subject.

As a result, an IR drug detection device user may detect whether a human subject is currently experiencing the intoxication effects of THC and distinguish that human subject from one that was previously experiencing the intoxication effects of THC. In a specific example implementation, the presently disclosed technology discriminates between an analyte, Δ-9-THC, and two closely related metabolites thereof (i.e., the hydroxyl-Δ9-THC and carboxy-Δ9-THC) using an IR bandwidth of 5.5-8.3 microns. While these three compounds have very similar chemical structures that differ only in terms of the functional groups attached to one carbon atom within the structures, as shown in FIG. 3, the presently disclosed technology can distinguish the chemical structures. For example, a significant drop (e.g., greater than 10%) in transmittance at about 5.7-5.8 microns bandwidth may indicate the presence of carboxy-Δ9-THC. Conversely, significant drops in transmittance at about 6.0-6.4 microns and 6.8-7.1 microns bandwidth may indicate the presence of the Δ9-THC and the hydroxyl-Δ9-THC, respectively. Still further, a significant drop in transmittance at about 7.6-8.0 microns may indicate the presence of Δ9-THC, hydroxyl-Δ9-THC, and carboxy-Δ9-THC. Analysis of these results can distinguish between psychoactive compounds in the test specimen and non-psychoactive metabolite compounds thereof. Further analysis of these results may distinguish individual psychoactive compounds, as well as relative concentrations of the psychoactive compounds.

FIG. 4 illustrates an example breath condensate collection device 400. The device 400 includes a holder 442 that selectively secures a specimen substrate 418 within the device 400. Further, the device 400 includes a mouthpiece 416 directed at a specific discrete location on the specimen substrate 418. The mouthpiece 416 allows a human subject to exhale breath into the device 400 and direct the subject's breath on the substrate 418, where a quantity of the subject's breath condenses on the specified discrete area of the substrate 418. The substrate 418 can then be tested using an IR detection device (e.g., devices 100, 200 of FIGS. 1A-2) to determine if the condensed breath contains any analytes, and in some cases a relative quantity of detected analyte(s) is determined. In other implementations, a face mask covering one or both of the mouth and nose may be used in place of the mouthpiece 416. In various implementations, the entire device 400, merely the substrate 418, or some subassembly thereof is selectively inserted into the IR detection device. In other implementations, the device 400 is incorporated as an integral part of the IR detection device. While device 400 is discussed in detail with regard to breath condensate, other bodily fluids could be similarly deposited on the substrate 418 using the device 400.

In general, the device 400 immobilizes a test specimen potentially containing one or more analyte(s) in a manner that facilitates detection of the analyte(s) by the IR detection device. In various implementations, the collection device 400 is handled in a manner that significantly reduces or altogether avoids contamination of the test specimen after collection from the human subject. The substrate 418 may be removable or permanently integrated with the device 400. Further, the device 400 may be removable or permanently integrated with the IR detection device.

In various implementations, the device 400 includes an indicator that provides an indication of adequate collected test specimen (e.g., it may incorporate a color changing material sensitive to moisture). Example composition materials for the substrate 418 include IR specimen cards, coupons, open-cell foams, swabs, pads, coated particulates, microspheres, tubes, and cuvettes, each of which may have high transparency in the IR range of interest for a specific application. The substrate 418 may also be composed of a polymeric material, such as polyethylene, polypropylene, and polytetrafluoroethylene (PTFE). In some implementations, the substrate 418 may be modified by biofunctionalization, plasma cutting, etching, milling, or another method to increase the substrate's affinity for analyte(s), or decrease the substrate's affinity for metabolites or other potentially interfering chemical compounds. Any suitable substrate 418 form factor may be used for the device 400.

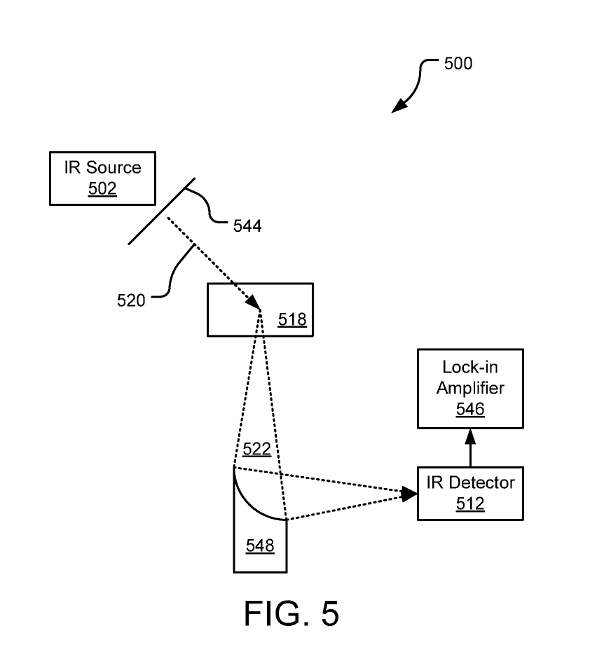

FIG. 5 illustrates a schematic of an example IR drug detection device 500. The device 500 includes an IR source 502, which may utilize any available IR generating technology and may include an array of multiple individual IR sources or a single IR source. In an example implementation, the IR source 502 is a tunable wavelength quantum cascade laser (QCL). The IR source 502 projects a source beam 520 through an optical chopper 544 (e.g., a variable frequency rotating disc chopper, a fixed-frequency tuning fork chopper, or optical shutters) to modulate the IR source 502 output intensity. The modulated source beam 520 impinges on a substrate 518 containing a test specimen (not shown). In various implementations, the IR source 502 includes additional source optics (not shown), which may steer, shape, filter, and/or disperse the light emitted from the IR source 502.

In various implementations, portions of the source beam 520 are reflected from the substrate 518, absorbed by the substrate 518, and/or transmitted through the substrate 518. In the depicted implementation, a portion of the source beam 520 is reflected from the substrate 518 to generate a reflected beam 522, which has a wavelength-intensity pattern (or spectral signature) commensurate with the test specimen and the substrate 518 and their interaction with the source beam 520. The reflected beam 522 is directed to parabolic mirror 548 (e.g., an off-axis gold parabolic mirror), which then focuses the reflected beam 522 on IR detector 512 (e.g., a mercury cadmium telluride (MCT) IR detector). In various implementations, the IR detector 512 includes additional detector optics (not shown), which may steer, shape, filter, and/or disperse the reflected beam 522 incoming to the IR detector 512. In other implementations, a transmitted beam (not shown) and/or absorbed thermal energy is utilized for IR drug detection in addition to or in lieu of the reflected beam 522 as described herein. A lock-in amplifier 546 may be used in conjunction with the optical chopper 544 to improve the signal-to-noise ratio of the signal detected by the IR detector 512.

In an example implementation, the IR source 502 is tuned to generate the source beam 520 with a wavelength approximately 6.15 μm (or 5.54 μm-6.77 μm) and an output power of approximately 50 mW (or 45 mW-55 mW). The optical chopper 544 operates at approximately 10 Hz (or 9 Hz-11 Hz) in an example absorbance implementation and approximately 400 Hz (or 360 Hz-440 Hz) in example transmittance or reflectance implementations. The parabolic mirror 548 has an effective focal length of approximately 50 mm (or 45 mm-55 mm) and a diameter of approximately 50 mm (or 45 mm-55 mm) in an example implementation.

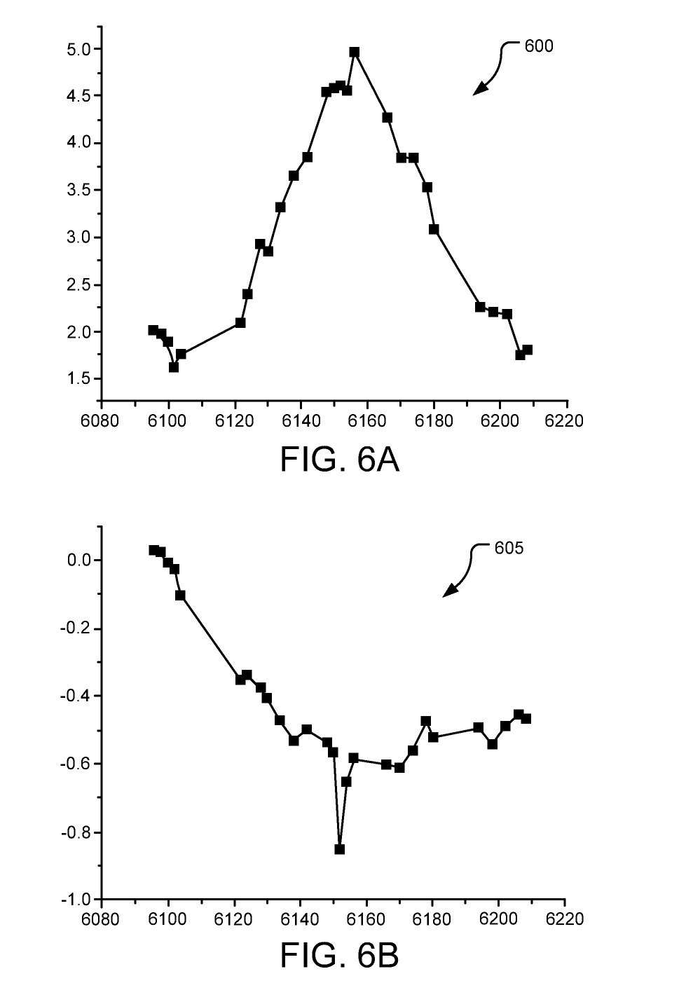

FIG. 6A is an example graph 600 of IR absorbance as a function of wavelength for a drug analyte. The graph 600 is generated as a result of using an IR detection device (see e.g., devices 100, 200 of FIGS. 1A-2) operating in an absorbance implementation. The graph 600 plots IR absorbance in absorbance units (a.u.) over wavelength in nanometers (nm). The graph 600 is compared with a control graph of the IR absorbance as a function of wavelength for a substrate only. Any differences between graph 600 and the control graph are compared with IR response characteristics of the analyte(s) to determine if the analyte(s) are present on the substrate. In an example implementation, the graph 600 is generated using approximately 20 micrograms of Δ-9-THC on a polyethylene IR specimen card.

FIG. 6B is an example graph 605 of IR reflectance as a function of wavelength for a drug analyte. The graph 605 is generated as a result of using an IR detection device (see e.g., devices 100, 200 of FIGS. 1A-2) operating in a reflectance implementation. The graph 605 plots IR reflectance fraction over wavelength in nanometers (nm). The graph 605 is compared with a control graph of the IR reflectance as a function of wavelength for a substrate only. Any differences between graph 605 and the control graph are compared with IR response characteristics of the analyte(s) to determine if the analyte(s) are present on the substrate. In an example implementation, the graph 605 is generated using approximately 20 micrograms of Δ-9-THC on a polyethylene IR specimen card.

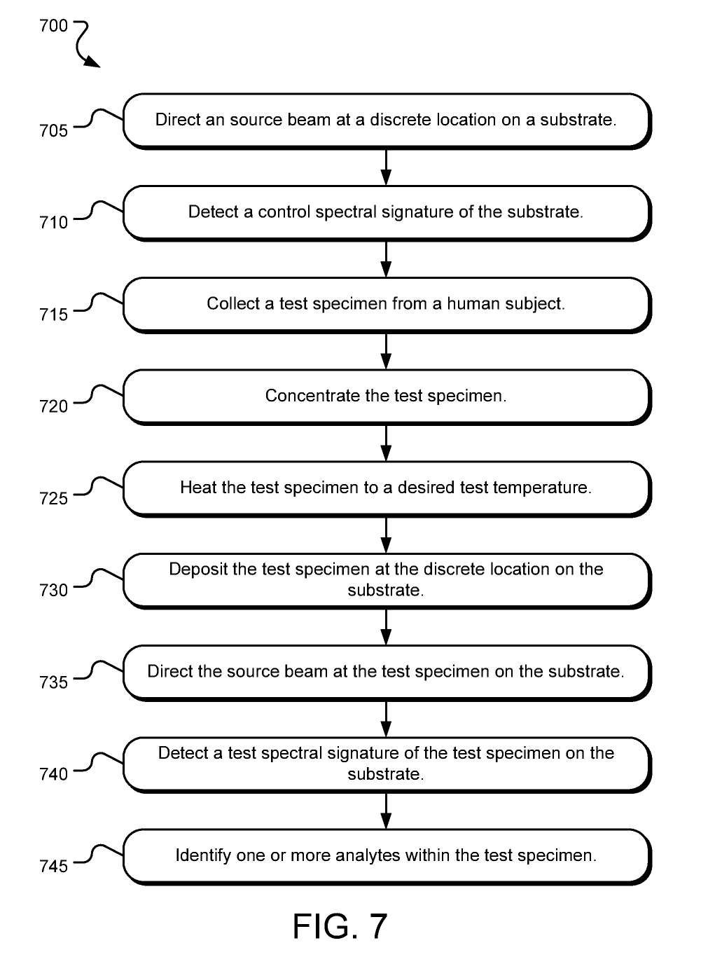

FIG. 7 illustrates example operations 700 for using an IR drug detection device to detect the presence of one or more analyte(s) in a test specimen. A directing operation 705 directs an IR source beam at a discrete location on a substrate. In an example implementation, the IR source 202 and source optics 204 of FIG. 2 perform the directing operation 705. A detection operation 710 detects a control spectral signature of the substrate. The detection operation 710 utilizes one or more portions of the source beam transmitted through the substrate, a portion of the source beam reflected from the substrate, and thermal emission from the substrate following interaction with the source beam. The control spectral signature is a test reading on a substrate to identify any chemical compounds preexisting on the substrate, for example. More specifically, the control spectral signature is used to distinguish spectral characteristics of the substrate from spectral characteristics of the analyte(s) in the test specimen. In an example implementation, the detector optics 208 and the IR detector 212 of FIG. 2 perform the detecting operation 710.

A collecting operation 715 collects the test specimen from a human subject. In various implementations, the test specimen is breath condensate, saliva, or other bodily fluids, for example. In various implementations, the human subject exhales breath onto the substrate via a breath collection device (see e.g., breath condensate collection device 400 of FIG. 4). More specifically, the mouthpiece 416 of FIG. 4 may perform the collecting operation 715 to collect one or both of breath condensate and saliva from the human subject. In various implementations, the substrate may be selectively installed and removed from the breath collection device for multiple uses or the breath collection device may be contiguous or a singular disposable apparatus. In some implementations, the substrate is sealed prior to use to prevent contamination. In other implementations, the substrate is sealed after use to preserve the substrate for evidentiary purposes.

A concentration operation 720 concentrates the test specimen prior to depositing the test specimen on the substrate in order to improve reliability and repeatability of the operations 700. In an example implementation, the concentration device 250 of FIG. 2 performs the concentration operation 720. In some implementations, the concentration operation 720 is omitted. A heating operation 725 heats the test specimen to a desired test temperature prior to detecting a spectral signature of the test specimen. In various implementations, the spectral signature may be best detected and/or distinguished from other spectral signatures at the test temperature. In an example implementation, the temperature control element 210 of FIG. 2 performs the heating operation 725.

A depositing operation 730 deposits the test specimen at the discrete location on the substrate. The substrate captures and holds the test specimen in place for detecting a spectral signature of the test specimen. In an example implementation, the mouthpiece 416 of FIG. 4 also performs the depositing operation 730 to direct the collected test specimen at the discrete location on the substrate. In various implementations, some or all of the collecting operation 715, the concentration operation 720, the heating operation 725, and the depositing operation 730 may be performed in the order depicted in FIG. 7, another order, simultaneously, omitting or adding operations, or any combination thereof.

A second directing operation 735 directs the source beam at the test specimen on the substrate. In an example implementation, the IR source 202 and source optics 204 of FIG. 2 also perform the second directing operation 735. A second detection operation 740 detects a spectral signature of the test specimen on the substrate. The second detection operation 740 utilizes one or more portions of the infrared beam transmitted through the test specimen and the substrate, a portion of the infrared beam reflected from the test specimen and the substrate, and thermal emission from the test specimen and the substrate following interaction with the source beam. The spectral signature combines the spectral signature of the test specimen and the substrate. In an example implementation, the detector optics 208 and the IR detector 212 of FIG. 2 also perform the second detecting operation 740.

An identification operation 745 identifies one or more analytes within the test specimen. The spectral signature is analyzed and compared to known characteristics of the analytes, as well as the control spectral signature. More specifically, the spectral signature may have bandwidth-specific characteristics that can identify and perhaps quantify analytes within the test specimen on the substrate, while taking into account the preexistence of any chemical compounds detected in the first detection operation 710 prior to outputting analyte detection results. In one implementation, the identification operation 745 identifies and distinguishes the analyte(s) from one or more metabolites thereof within the test specimen. In another implementation, the identification operation 745 identifies and distinguishes psychoactive tetrahydrocannabinol compounds from non-psychoactive tetrahydrocannabinol compounds within the test specimen.

The embodiments of the invention described herein are implemented as logical steps in one or more computer systems. The logical operations of the present invention are implemented (1) as a sequence of processor-implemented steps executing in one or more computer systems and (2) as interconnected machine or circuit modules within one or more computer systems. The implementation is a matter of choice, dependent on the performance requirements of the computer system implementing the invention. Accordingly, the logical operations making up the embodiments of the invention described herein are referred to variously as operations, steps, objects, or modules. Furthermore, it should be understood that logical operations may be performed in any order, adding or omitting operation as desired, unless explicitly claimed otherwise or a specific order is inherently necessitated by the claim language.

The above specification, examples, and data provide a complete description of the structure and use of exemplary embodiments of the invention. Since many embodiments of the invention can be made without departing from the spirit and scope of the invention, the invention resides in the claims hereinafter appended. Furthermore, structural features of the different embodiments may be combined in yet another embodiment without departing from the recited claims.

What is claimed is:

1. An infrared drug detector comprising:
a bodily fluid collector directed at a discrete location on a substrate and configured to deposit a bodily fluid specimen on the substrate;
an infrared source directed at the discrete location on the substrate and configured to emit a source beam at the bodily fluid specimen; and
an infrared detector configured to receive a spectral signature of the bodily fluid specimen following interaction of the bodily fluid specimen with the infrared source beam to detect the presence of an analyte within the bodily fluid specimen.

2. The infrared drug detector of claim 1, further comprising:
a substrate holder configured to secure the substrate in a desired position relative to the bodily fluid collector prior to deposition of the bodily fluid specimen on the substrate.

3. The infrared drug detector of claim 1, further comprising:
source optics configured to direct the source beam emitted from the infrared source to the substrate; and
detector optics configured to direct the spectral signature of the bodily fluid specimen to the infrared detector.

4. The infrared drug detector of claim 3, wherein the source optics include a mechanical chopper and the detector optics include a parabolic mirror.

5. The infrared drug detector of claim 1, further comprising:
a temperature control element configured to achieve a desired detection temperature of the bodily fluid specimen on the substrate.

6. The infrared drug detector of claim 1, further comprising:
a concentration device configured to concentrate the bodily fluid specimen at the discrete location on the substrate.

7. The infrared drug detector of claim 1, wherein the detector optics are capable of distinguishing one or more analytes from one or more metabolites thereof within the bodily fluid specimen.

8. The infrared drug detector of claim 1, wherein the detector optics are capable of distinguishing psychoactive tetrahydrocannabinol compounds from non-psychoactive tetrahydrocannabinol compounds within the bodily fluid specimen.

9. The infrared drug detector of claim 1, wherein the spectral signal is embodied in one or more of an infrared beam transmitted through the substrate, an infrared beam reflected from the substrate, and thermal emission from the substrate.

10. The infrared drug detector of claim 1, wherein the infrared detector is further configured to quantify the presence of the analyte within the bodily fluid specimen.

11. A method comprising:
depositing a bodily fluid specimen at a discrete location on a substrate;
directing an infrared source beam at the discrete location on the substrate;
detecting a spectral signature of the bodily fluid specimen on the substrate following interaction of the bodily fluid specimen with the infrared source beam; and
identifying one or more analytes within the bodily fluid specimen using the detected spectral signature.

12. The method of claim 11, further comprising:
achieving a desired test temperature of the bodily fluid specimen on the substrate prior to detecting the spectral signature of the bodily fluid specimen.

13. The method of claim 11, further comprising:
pre-concentrating the bodily fluid specimen at the discrete location on the substrate prior to detecting a spectral signature of the bodily fluid specimen.

14. The method of claim 11, wherein the identifying operation includes distinguishing the one or more analytes from one or more metabolites thereof within the bodily fluid specimen.

15. The method of claim 11, wherein the identifying operation includes distinguishing psychoactive tetrahydrocannabinol compounds from non-psychoactive tetrahydrocannabinol compounds within the bodily fluid specimen.

16. The method of claim 11, wherein the spectral signal is embodied in one or more of an infrared beam transmitted through the substrate, an infrared beam reflected from the substrate, and thermal emission from the substrate.

17. The method of claim 11, wherein the detecting operation is performed non-destructively on the bodily fluid specimen.

18. The method of claim 11, wherein the identifying operation includes quantifying the one or more analytes within the bodily fluid specimen.

19. A method of infrared drug detection comprising:
directing an infrared source beam at a discrete location on a substrate;
detecting a control spectral signature of the substrate following interaction of the substrate with the infrared source beam;
depositing a bodily fluid specimen at the discrete location on the substrate following detection of the control spectral signature of the substrate;
directing the infrared source beam at the discrete location on the substrate following deposition of the bodily fluid specimen;
detecting a condensate spectral signature of the bodily fluid specimen on the substrate following interaction of the bodily fluid specimen with the source beam; and
identifying one or more analytes within the bodily fluid specimen using the detected spectral signature.

20. The method of claim 19, wherein the identifying operation includes distinguishing the one or more analytes from one or more metabolites thereof within the bodily fluid specimen.

21. The method of claim 19, wherein the identifying operation includes distinguishing psychoactive tetrahydrocannabinol compounds from non-psychoactive tetrahydrocannabinol compounds within the bodily fluid specimen.

22. The method of claim 19, wherein the spectral signal is embodied in one or more of an infrared beam transmitted through the substrate, an infrared beam reflected from the substrate, and thermal emission from the substrate.
